(19) United States
(12) Patent Application Publication
(10) Pub. No.: US 2006/0025723 A1
(43) Pub. Date: Feb. 2, 2006

(54) Antibacterial chest tube, surgical drain, port or access line securing device

(76) Inventor: V. Joseph Ballarini, Philadelphia, PA (US)

Correspondence Address: Robert J. Ballarini, 8242 Shawnee Street, Philadelphia, PA, 19118, US

(21) Appl. No.: 11/191,865

(22) Filed: Jul. 28, 2005

Related U.S. Application Data
(60) Provisional application No. 60/591,810, filed on Jul. 28, 2004.

(51) Int. Cl. A61M 5/32

(52) U.S. Cl. 604/180; 606/232

(57) ABSTRACT

A device to secure a tube inserted into a patient is provided. The device has a main body, a tube passage defining an opening through the main body and is integral with the main body, and a tube securing element that is associated with the passage. The device is fixed in place by securing the main body to the person of a patient. The present invention also relates to a method for securing a tube inserted into a patient.

### CROSS REFERENCE TO RELATED APPLICATIONS

This application claims the benefit of U.S. Provisional Application No. 60/591,810, filed Jul. 28, 2004, which is incorporated by reference as if fully set forth.

### FIELD OF THE INVENTION

The present invention relates to a device and method for securing tubes inserted into a patient.

### BACKGROUND

Chest tube thoracostomy is a medical procedure performed to drain fluid, blood, or air from the space around the lungs. Certain diseases, e.g. pneumonia and cancer cause a pleural effusion or an excess amount of fluid or blood to build up in the space around the lungs. Severe injuries/infections of the chest wall can also cause bleeding around the lungs. Sometimes, the lung can be accidentally punctured allowing air to gather outside the lung, causing a pneumothorax or chest collapse. Chest tube thoracostomy involves feeding a hollow flexible tube between a patient's ribs and into the chest to drain fluid or air from around the lungs. A suction machine is often attached to the tube to help with drainage. The tube remains in the patient's chest until all or most of the air or fluid has been drained. This procedure generally lasts a few days. A stitch (suture), a safety pin and/or adhesive tape is often used to keep the tube in place. These drains have potential complications such as complications due to disconnection; introduction of infection from outside via the drain track; and erosion by the drain of adjacent tissues. Sutures also slip since they are only wrapped around a cylindrical tube and can and do slip and slide Furthermore, a patient's movement is restricted due to the tube being effectively sewn to his or her body. Therefore, there is a need for an improved system for securing a tube inserted into a patient.

### SUMMARY

The present invention relates to a device to secure a tube inserted into a patient, the device comprising a main body having first and second portions; a tube passage defining an opening through the main body and integral therewith; and a tube securing element, associated with the passage, wherein the device is fixed in place by securing the main body to a patient's body.

The present invention also relates to a method for securing a tube inserted into a patient comprising the steps of: attaching a tube securing device to a patient's body at an incision point; inserting a tube through a tube passage of the device and into the incision; and securing the tube to the device.

### BRIEF DESCRIPTION OF THE DRAWINGS

Further characteristics and advantages of the invention shall become clearer from the following description of its preferred embodiments, given with reference to the attached drawings. In such drawings FIGS. 1 to 19 concern a device for securing a tube in a patient; more specifically:

FIG. 1 is a perspective view of a first embodiment of the device of the present invention.

FIG. 2 is a bottom plan view of the device of FIG. 1.

FIG. 3 is a top plan view of the device of FIG. 1.

FIG. 4 is a side elevational view of the device of FIG. 1.

FIG. 5 is a perspective view of a variant of the device of FIG. 1.

FIG. 6 is a side elevational view of the device of FIG. 5.

FIG. 7 is a perspective view of an alternate embodiment of the device of the present invention.

FIGS. 8a and 8b are top plan views of the device of FIG. 7.

FIG. 9 is a bottom plan view of the device of FIG. 7.

FIG. 10 is a second bottom plan view of the device of FIG. 7 having an adhesive layer.

FIG. 11 is an elevated side view of the device of FIG. 7 depicting a tube therein.

FIG. 12 is a section view of the device of FIG. 7 taken along line 12-12.

FIG. 13 is a partial section view of the device of FIG. 7 taken along line 13-13.

FIG. 14 is an exploded view of the device of FIG. 7.

FIG. 15 is a perspective view of a variant of the device of FIG. 7.

FIG. 16 is an elevated side view of the variant of FIG. 15.

FIG. 17 is a perspective view of a further alternate embodiment of the device of the present invention.

FIG. 18 is a partial section view of the device of FIG. 17.

FIG. 19 shows the device of the present invention in use with a patient.

### DETAILED DESCRIPTION OF THE PREFERRED EMBODIMENTS

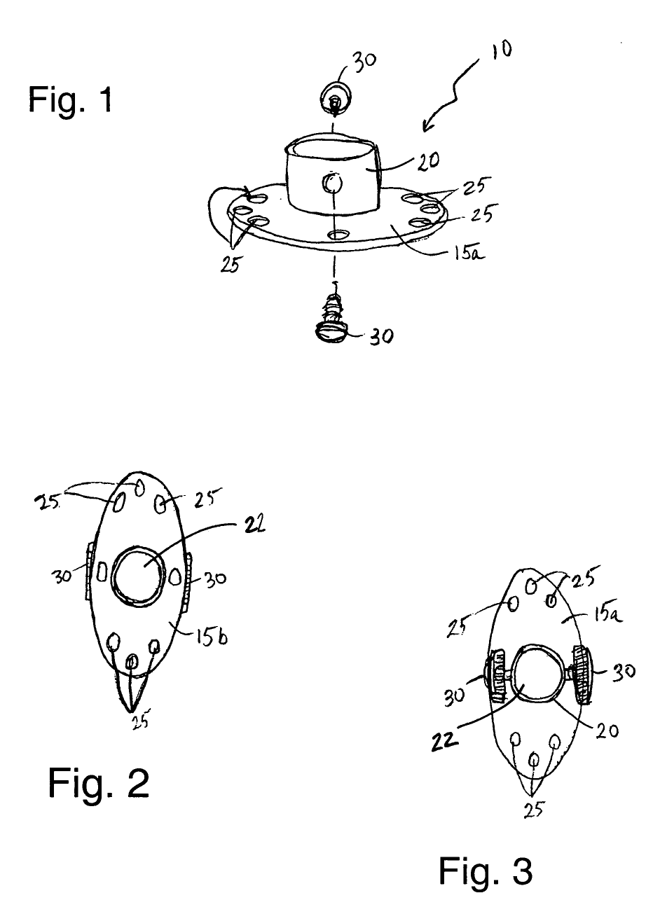

FIGS. 1-4 show a first embodiment of the device 10 of the present invention. The device 10 comprises a main body 15 having top and bottom portions 15a, 15b. The main body 15 comprises through holes or suture holes 25 about its periphery. The bottom portion 15b of the main body 15 may also comprise an adhesive layer 55 (shown in FIG. 10). A hollow tube passage 20 is integral with the main body 15 and extends perpendicularly from the top portion 15a of the main body and has a generally cylindrical shape. The tube passage 20 defines a through aperture 22 through the main body 15 which receives a tube 90. The tube passage also comprises one or more openings 35 that accept fasteners such as screws or bolts 30 that extend into the opening 22. In this embodiment, the screws or bolts 30 act as tube securing elements. The openings 35 are located in the cylindrical side wall of the passage 20.

The device 10 is made of a plastic material which is preferably an antimicrobial material which reduces the risk of bacterial infection to the open epidermis and underlying tissue. Such material also offers greater comfort to the patient, and greater flexibility to the physician. Although antimicrobial plastic is a preferred material for the device 10, it should also be noted that the device 10 can be made from metal, e.g. surgical stainless steel, titanium, or antimicrobial metal alloys containing copper or silver.

The device 10 of the present invention can be made in both adult and pediatric sizes. Main body 15 is an oval-shaped piece of plastic of varying thickness roughly 0.10 to 0.25 inches thick having 3 holes 25 at either end (12 & 6 o'clock) and 1 hole on each top lateral (3 & 9 o'clock) for passage of sutures and anchoring to skin. The bottom portion 15b has an adhesive layer 55 covered by a film 60 to prevent sticking unless removed.

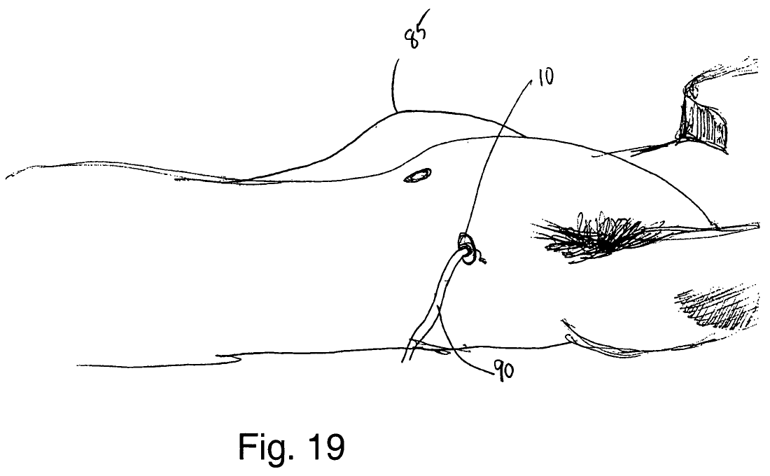

In use, the device 10 is placed on a tube, port or access line prior to it being inserted into the patient being treated, or in most cases can be added after the tube 90 (FIG. 19) is in place within the body of a patient 85. Once the chest tube 90 is in place, the device 10 is guided up the inserted chest tube 90, to the surface of the skin. The bottom portion 15b of the main body 15 contacts the skin and is sutured by passing sutures through suture holes 25 of the main body 15 effectively securing the device 10 to the patient 85. Alternatively, the device 10 can be secured to the patient 85 by adhesive layer 55. Alternatively, as mentioned the clamp can be secured to the surface of the patient's skin by an antibacterial adhesive. The device 10 can also have an adhesive layer 55 on its bottom portion 15b. The adhesive layer 55 is covered by a removable film 60 to expose the adhesive layer 55 once the tube 90 is in place. The use of an adhesive layer 55 is less invasive and does not require a physician to suture causing greater discomfort. The skin should be prepared with topical skin prep agent prior to attaching the main body 15 of the device 10. The device 10 can be removed from the patient by peeling it off or by the use of a tape releasing agent. A combination of sutures and adhesive can also be used. After suturing or adhering the device 10 into place, the chest tube 90, is finely adjusted and securing elements 30 are tightened to contact the tube 90 in the opening 22 of the tube passage 20 securing it firmly in place.

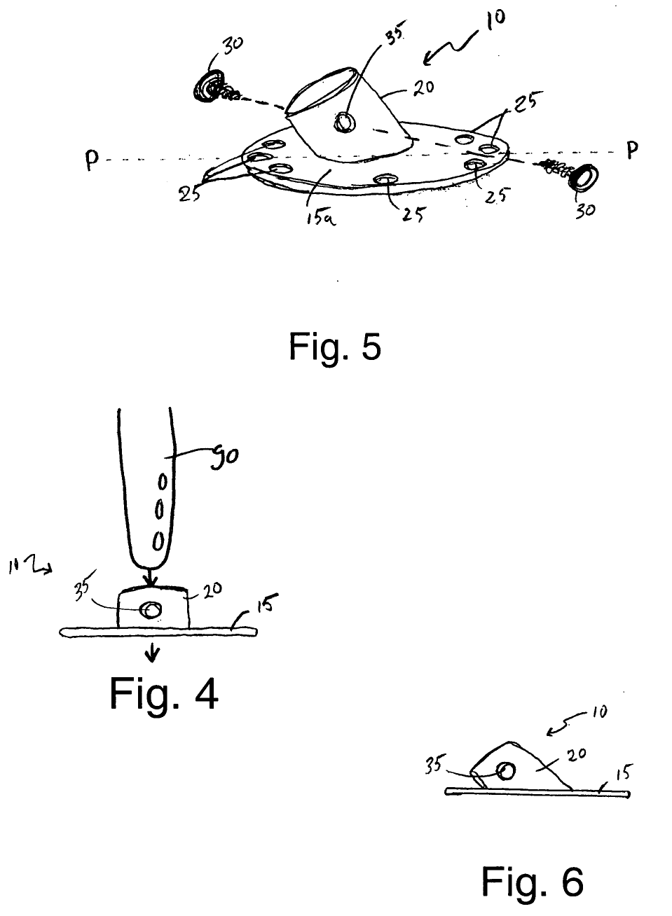

A variant of the device 10 of FIGS. 1-4 is shown in 5 and 6. As shown in FIG. 5, the tube passage 20 extends at an angle from the lateral plane P of the main body 15. Such positioning of the tube passage 20, allows for greater flexibility in positioning the tube 90 and apparatus associated therewith, e.g. pumps, collection bags, etc. The angle of the tube can vary between 17 to 170° depending on the position of the tube insertion.

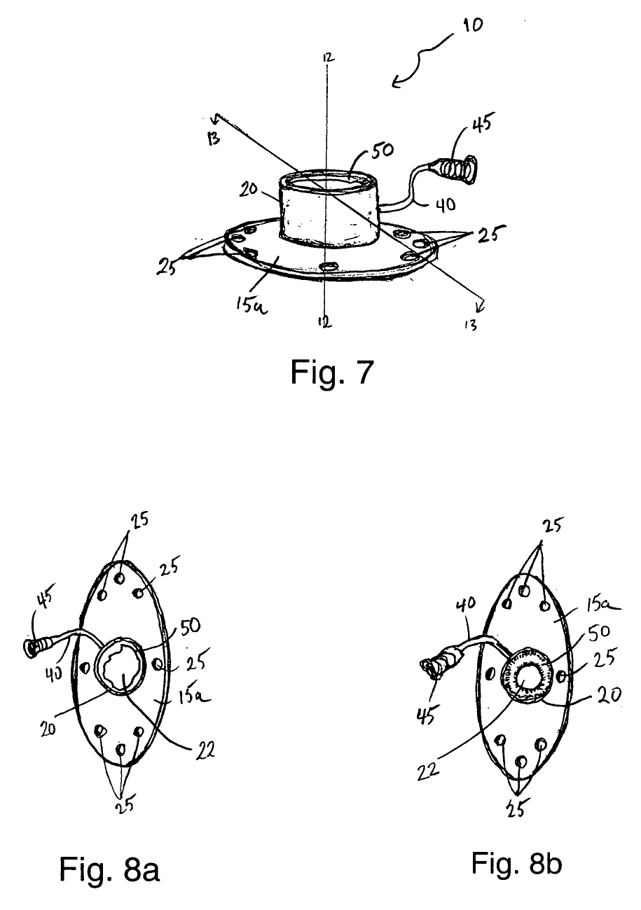

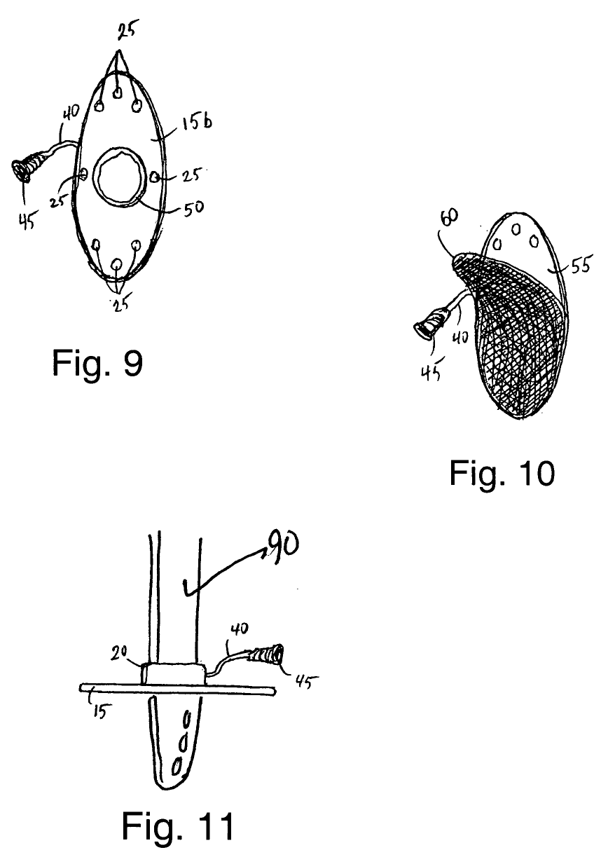

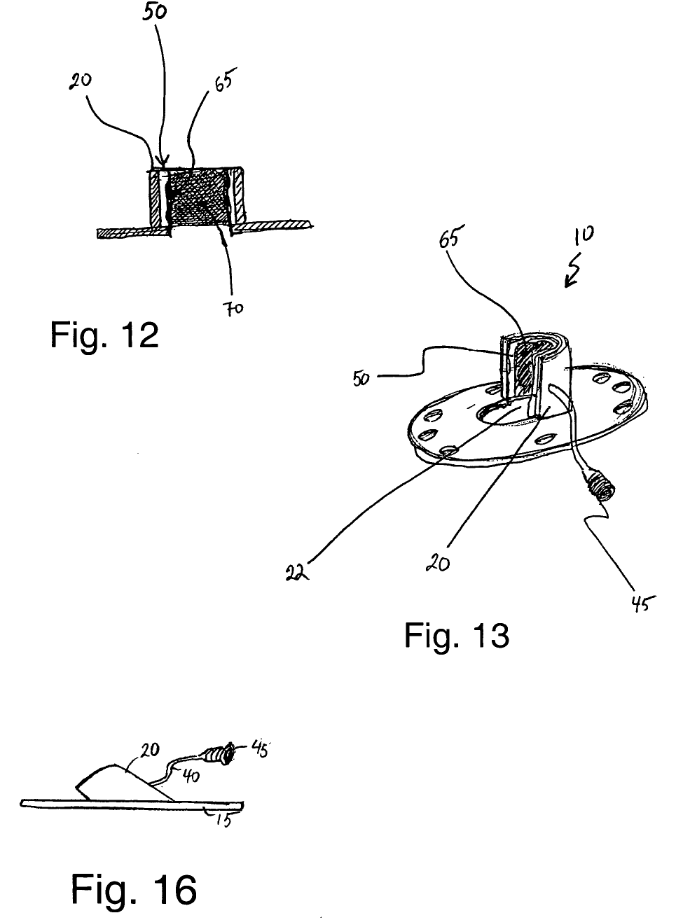

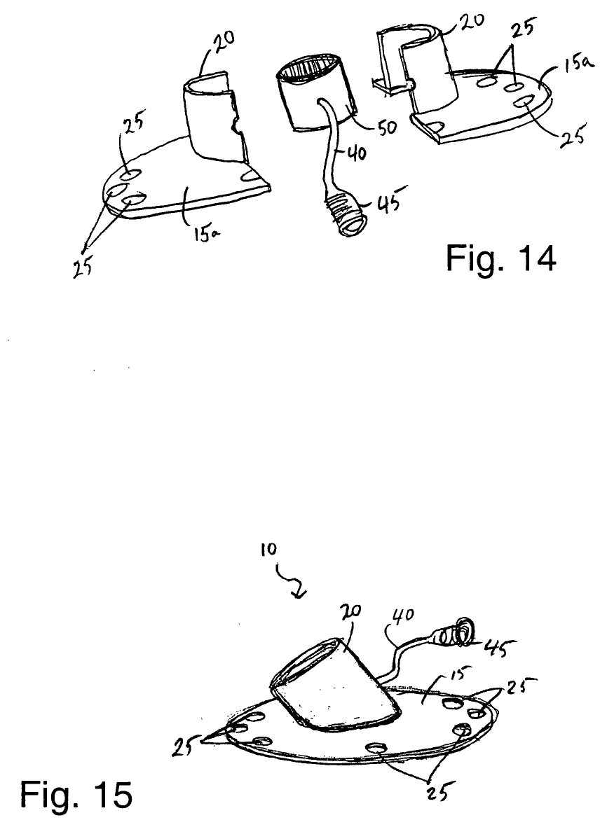

A second embodiment of the device 10 is shown in FIGS. 7-15. In this embodiment, the tube passage 20 is fitted with an inner sleeve 50. In this embodiment, the inner sleeve 50 acts as tube a securing element. The sleeve is in communication with a port 40 and an interface 45. Interface 45 comprises a variable valve which permits air or water (or other suitable gasses or liquids) to enter the sleeve 50. The valve also allows air or water to exit the sleeve 50. When a tube is in place within the tube passage 20 of the device 10, the sleeve 50 is inflated with air or water through the interface 45 and port 40 until the tube is secure within the device. FIG. 8a shows the sleeve 50 in a deflated or unexpanded state. FIG. 8b shows the sleeve 50 inflated or expanded. To remove or adjust the tube 90 the valve of the interface 45 is opened to remove the air or water from the ring. Air or water can be introduced to the sleeve 50 by a syringe or other suitable means. The inner most portion of the inflatable sleeve that touches the tube is made of a synthetic material that will keep the tube 90 from slipping.

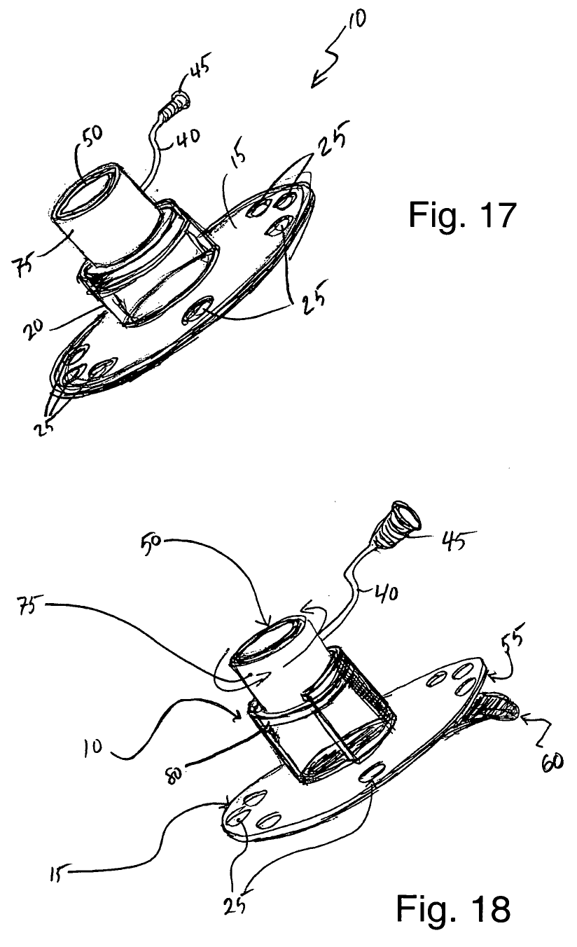

A third embodiment, similar to the second embodiment above is shown in FIGS. 17 and 18. In this embodiment, sleeve 50 is arranged within a clamp port 75. The clamp port 75, comprises a circumferential protrusion 80 which matingly engages a circumferential groove 82 of the tube passage 20. This allows the clamp port 75 and sleeve to rotate in the device. The device 10 according to this embodiment can be used when rotation of the tube is necessary.

The present invention provides a patient greater comfort, and ease of movement while offering the physician the ability to adjust the placement of the tube, port or line without having to cut out the original suture and start over. Once the device is in place, it does not have to be removed until the physician decides to remove the chest, port or access line. It become a semi-permanent port & clamp for holding inserted thoracostomy tube, any port, any surgical drain or any access line in place. It is contemplated that the device of the present invention can be dimensioned for use on any part of a patient's body.

The torque and drag on the skin from the suture will be equally distributed, rather than pinpoint drag on an open flap of skin. If the embodiment using adhesive is employed, the torque and drag is distributed along the entire profile of the main body of the device. This results in a greater comfort for the patient. Since the tube is not sewn to the patient, the chest tube, port or access line becomes adjustable. Furthermore, the risk of infection is greatly reduced because the incision is completely covered with an antibacterial device instead of a gauze.

What is claimed is:

1. A device to secure a tube inserted into a patient's body, the device comprising:
a main body having first and second portions;
a tube passage extending from the first portion of the main body and integral therewith defining an opening through the main body; and
at least one tube securing element, associated with the passage, wherein the device is fixed in place by securing the main body to the patient's body.

2. The device of claim 1, wherein the main body further comprises a plurality of suture openings about its periphery.

3. The device of claim 2, wherein the device main body is secured to a patient's body by suturing.

4. The device of claim 1, wherein the main body further comprises an adhesive layer on its second portion.

5. The device of claim 4, wherein the device is secured to a patient's body by the adhesive layer.

6. The device of claim 1, wherein the tube securing element comprises a threaded member passing through an opening in the tube passage.

7. The device of claim 6, wherein the threaded member is manually adjustable.

8. The device of claim 1, wherein the tube securing element comprises an inner sleeve disposed within the tube passage.

9. The device of claim 8, wherein the inner sleeve is expandable to secure a tube to the device.

10. The device of claim 9, wherein the inner sleeve is expanded pneumatically.

11. The device of claim 9, wherein the inner sleeve is expanded hydraulically.

12. The device of claim 9, further comprising a clamp port, wherein the inner sleeve is arranged within the clamp port.

13. The device of claim 12, wherein the clamp port comprises a circumferential projection matingly engagable with a circumferential groove of the tube passage.

14. The device of claim 13, wherein rotation of the tube is permitted when the projection is engaged within the groove.

15. The device of claim 1, wherein the tube passage extends perpendicularly to a plane of the main body.

16. The device of claim 1, wherein the tube passage extends at an angle less than 90° to a plane of the main body.

17. A method for securing a tube inserted in a patient's body, the method comprising the steps of:
a) providing a device having:
a main body having first and second portions;
a tube passage extending from the first portion of the main body and integral therewith defining an opening through the main body; and
a tube securing element, associated with the passage;
b) securing the device to the patient's body; and
c) securing the tube to the device.

18. A device to secure a tube inserted into a patient's body, the device comprising:
a main body having first and second portions;
a tube passage extending from the first portion of the main body and integral therewith defining an opening through the main body; and
means for securing the tube, associated with the passage, wherein the device is fixed in place by securing the main body to the patient's body.
